(19) United States
(12) Patent Application Publication
(10) Pub. No.: US 2009/0316854 A1
(43) Pub. Date: Dec. 24, 2009

(54) Multi-modality system for screening, imaging and diagnosis in dense compressive media and method of use thereof

(76) Inventors: Aly M. Ismail, Irvine, CA (US); Khaled N. Satama, Troy, NY (US)

Correspondence Address: LAW OFFICES OF ROBERT HART, 28 EAST JACKSON BUILDING, 10TH FLOOR, SUITE H528, CHICAGO, IL, 60604, US

(21) Appl. No.: 12/214,968

(22) Filed: Jun. 23, 2008

(51) Int. Cl. A61B 6/00; A61B 8/00

(52) U.S. Cl. 378/4; 600/437

(57) ABSTRACT

A multi-modality system and method for performing screening/detection, imaging and diagnosis/characterization of materials and objects in dense compressive media, such as in medical soft tissue applications, is disclosed. Medical tissue applications include but are not limited to the detection and diagnosis of breast tumors. Generally, the present invention involves coupling X-ray mammography screening devices and methods with a system and method for further, real-time diagnosis of the X-ray results comprising an ultrasound subsystem for exciting target tissues and a microwave subsystem for measuring the response, imaging and diagnosing the target tissues.

### CROSS REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of Invention

The present invention relates generally to the field of imaging in dense compressive media, and more particularly to a novel system and method of use thereof for imaging in medical soft tissue applications such as dermatology, orthopedics and bone fractures, and breast tumor scanning/detection and diagnosis/characterization.

According to the U.S. National Library of Medicine and the National Institutes of Health, one in eight women will be diagnosed with breast cancer. One in sixteen women will die prematurely due to breast cancer. Breast cancer is more easily treated and often curable if it is discovered early. Breast cancer stages range from 0 to IV. The higher the stage number, the more advanced the cancer. According to the American Cancer Society (ACS), the 5-year survival rates for persons with breast cancer that is appropriately treated are as follows: 100% for Stage 0, 100% for Stage I, 92% for Stage IIA, 81% for Stage IIB, 67% for Stage IIIA, 54% for Stage IIIB, and 20% for Stage IV. Clearly, early detection is the primary factor in the successful treatment of breast cancer. Early breast cancer usually does not cause symptoms, therefore accentuating the importance of early detection and diagnosis devices and methods.

2. Discussion of Related Art

The usefulness of methods and/or devices to perform breast cancer detection is well recognized. A variety of related art methods and/or devices are directed to the problem. However, each related art method and/or device possesses significant disadvantages.

The principal methods of detecting breast cancer are clinical physical examination, self-examination, and X-ray mammography. Efforts have been made to develop alternative solutions to the problem of breast cancer detection and diagnosis, including magnetic resonance imaging (MRI), ultrasound, and microwave radar imaging.

In a clinical physical examination, a doctor performs a tactile physical examination of the breasts, armpits, and the neck and chest area. The physical examination is intended to discover lumps indicative of cancer. However, the clinical physical examination cannot identify the nature of the lump and lacks the sensitivity or resolution of other methods.

The breast self-examination is essentially the same as the clinical physical examination, but it is performed by the subject outside of the clinical environment. The breast self-examination is similar in benefit and limitation to the clinical physical examination.

X-ray mammography is currently the only FDA-certified early breast cancer screening technology. X-ray mammography can detect breast tumors before they can be detected by a physical examination. One breast at a time is rested on a flat surface that contains an X-ray detection media; typically a film exposure plate or digital imaging modality such as semiconductor detectors. A device called a compressor is pressed firmly against the breast to flatten the breast tissue. The patient holds her breath while a series of X-ray images are taken from several angles. While X-ray mammography is an effective screening method, it is not the most effective method for early-stage cancer detection. In a study, by Kuhl, et al. below, only 52 percent of high-grade ductal carcinoma in situ (DCIS), the form most likely to develop into invasive cancer, were detected by X-ray mammography. “MRI for Diagnosis of Pure Ductal Carcinoma In Situ: A Prospective Observational Study,” Christiane Kuhl, et al., The Lancet, vol. 370, issue 9586, 11 Aug. 2007, pages 485-492. While X-ray mammography is an effective screening tool, it is not an effective diagnostic tool. Initial mammographic images are not sufficient to distinguish benign from malignant tumors with certainty. Therefore, other diagnostic studies, such as MRI or biopsy, are often recommended in the event of a mammographic finding. Further, false positive indications are common in X-ray mammography screening. There is a 30 percent chance of a woman having a false positive mammogram at some point between the ages of 40 and 49. These false positive indications lead to further diagnostic studies, including unnecessary biopsies. “Mammography,” Radiology Info, Radiological Society of North America (RSNA), pp. 6-7, 2006.

Due to the limitations and disadvantages of these methods, there exists an on-going search for other effective methodologies. Magnetic resonance imaging, ultrasound, and microwave radar are solutions of interest.

Magnetic resonance imaging employs powerful magnets and radio waves to generate images inside the body. The MRI magnetic field polarizes the magnetic moment of hydrogen atoms in the body. When properly tuned radio waves are then transmitted through the body, they are differentially absorbed depending on the types of tissue encountered. The resulting radio signal can thus often distinguish healthy versus cancerous tissue. MRI represents a substantial improvement over X-ray mammography in terms of early screening, detecting 98 percent of high-grade DCIS compared with 52 percent detection by X-ray mammography, as noted in Kuhl, et al., “MRI for Diagnosis of Pure Ductal Carcinoma In Situ: A Prospective Observational Study,” Christiane Kuhl, et al., The Lancet, vol. 370, issue 9586, 11 Aug. 2007, pages 485-492.

While MRI offers improved screening accuracy over X-ray mammography, it also has significant disadvantages. Many patients find the MRI procedure uncomfortable. The patient may be required to fast from four to six hours prior to the scan. Then, the patient lies on a narrow table which slides into the center of the MRI scanner. The MRI machine may induce anxiety in patients with a fear of confined spaces. Further, the MRI machine produces loud percussive and buzzing noises which may be disconcerting to the patient. Finally, because several sets of images are required, each taking from two to fifteen minutes, the patient must be exposed to the MRI environment for an hour or longer. The patient is required to lie motionless for this long period of time because movement can blur MRI images and cause errors. In addition, because the magnet is very strong, certain types of metal can cause significant errors in the images, and the strong magnetic field created during an MRI can interfere with certain medical implants. Persons with pacemakers or other metallic objects in the body, such as ear implants, brain aneurism clips, artificial heart valves, vascular stents and artificial joints should not be exposed to MRI. Finally, the high cost of procuring and operating an MRI machine, and the lack of technicians skilled in reading breast MRIs present additional disadvantages to its use.

Research has turned to consideration of non-invasive ultrasound methods for screening and diagnosis, utilizing acoustic means for both excitation of the tissues and for measurement and imaging of the excited tissues. These methods utilize focused, oscillating high-frequency ultrasound input waves having slightly different frequencies to excite the target tissue at the intersection of the beams. These input waves propagate and interact producing a series of harmonic waves. One resultant harmonic is a low-frequency wave resulting from the cancellation of the high-frequency components of the input waves, generally known as the beat frequency. This low-frequency harmonic component produces a force that excites the target tissue or other target object for that matter. A publication by Alizad, et al., discusses one such acoustic method wherein a hydrophone is employed to detect the acoustic waves generated by the motion induced in the tissue. The detected acoustic waves are processed into imaging information. A. Alizad, M. Fatemi, L. E. Wold and J. F. Greenleaf, “Performance of Vibro-Acoustography in Detecting Microcalcifications in Excised Human Breast Tissue: A Study of 74 Tissue Samples,” IEEE Trans. Med. Imaging., vol. 23, pp. 307-312, March 2004. Hynyen, et al., U.S. Pat. No. 6,984,209 discloses another acoustic method which incorporates a pulse-echo ultrasound transceiver to perform the measurement and imaging function. Methods that rely upon acoustic measurement alone are disadvantaged by noise, contrast and speckle limitations, and by the necessity to trade off low-frequency penetration against high-frequency resolution.

Microwave detection methods offer a factor of five improvement in detection sensitivity and diagnostic capacity over ultrasound methods. J. E. Joy, E. E. Penhoet and D. B. Petitti, “Saving Women's Lives: Strategies for Improving Breast Cancer Detection and Diagnosis,” Institute of Medicine and National Research Council, ISBN: 0-309-53209-4, 2005. Ultrasound methods rely on the measurement of variations in the mechanical properties of benign tissue and cancerous tumors, which are not large. On the other hand, microwave methods take advantage of the difference in dielectric constants associated with the water content of benign tissue and cancerous tumors, which vary dramatically. Therefore, research is turning to consideration of microwave radar devices and methods for soft tissue imaging. Microwave imaging offers a low-stress, low risk solution; requiring short exposure periods without the dangers or discomforts associated with X-ray mammography or MRI. The scientific principles are defined and experimental demonstration discussed in a publication by Li, et al., “Microwave Imaging via Space-Time Beamforming: Experimental Investigation of Tumor Detection in Multilayer Breast Phantoms,” Xu Li, et al., IEEE Transactions on Microwave Theory and Techniques, Vol. 52, No. 8, August 2004. Li, et al., experimentally demonstrated the effectiveness of radar imaging principles in breast tumor detection applications employing a two-dimensional scanning methodology to synthesize a two dimensional antenna array. However, imaging methods that rely on microwave alone are disadvantaged by the necessity to trade off low-frequency penetration against high-frequency resolution.

Because of the limitations associated with each individual screening, imaging and diagnosis method, research is considering combining multiple imaging modalities.

A study by the American College of Radiation Imaging Network (ACRIN) validated the benefit of combining multiple modalities in breast cancer screening. In the ACRIN 6666 trial, diagnostic yield was compared for screening with ultrasound plus mammography versus mammography alone. This study concluded that adding a single screening ultrasound to mammography increased diagnostic accuracy from 78% to 91% as compared to resulted mammography alone. However, the study also indicated an increase in the number of false positives from 1 in every 40 women in the study for mammography alone to 1 in every 10 in the combination of mammography and ultrasound screening. W. A. Berg, et al., “Combined Screening With Ultrasound and Mammography vs Mammography Alone in Women at Elevated Risk of Breast Cancer,” Journal of the American Medical Association (JAMA), vol. 299 No.18, pp. 2151-2163, May 14, 2008. While this study demonstrated promise applied to the screening function, the low contrast, high noise characteristics of the ultrasound system contribute to false positive frequency and limited diagnostic capability.

Rosner, et al., U.S. Patent Application No. 2007/0276240 discloses a system which uses both acoustic and microwave methods for imaging. The ultrasound subsystem transmits ultrasound waves into the target and receives the echo. The microwave subsystem transmits a microwave signal into the target and receives the reflection. The ultrasound and microwave modalities operate independently. This simple integration of two modalities does not take advantage of the physical interaction of the ultrasound and microwave modalities. Therefore, this combined system possesses the disadvantages of each subsystem. It is difficult to concurrently achieve high penetration, high resolution, fast scanning and high contrast using either subsystem alone.

### BRIEF SUMMARY OF THE INVENTION

In view of the foregoing disadvantages inherent in the known devices and methods in the related art, the present invention provides a novel multi-modality system and method for performing screening/detection, imaging and diagnosis/characterization of materials and objects in dense compressive media, particularly but not exclusively in medical soft tissue applications. Specifically, the present invention involves coupling an X-ray screening device with a system and method for further diagnosis and imaging based upon the X-ray results comprising an ultrasound subsystem for exciting target tissues and a microwave subsystem for measuring the response and imaging the target tissues, without the disadvantages of the related art systems and methods.

The present invention integrates the demonstrated screening attributes of an X-ray mammography screening modality with the imaging and diagnostic advantages of integrated ultrasound and microwave modalities. The present invention involves a true hybrid integration of the ultrasound and microwave modalities, taking advantage of the best attributes of each subsystem modality. The superior resolution and focus characteristics of a plurality of focused high-frequency ultrasound input waves and the superior penetration and displacement force characteristics of the resultant low-frequency ultrasound harmonics are employed to excite Doppler displacements of materials in the target breast. At the same time, the superior penetration and high diagnostic contrast capabilities of the microwave modality are employed to perform the diagnosis an imaging function. Integration of the hybrid ultrasound-plus-microwave subsystems with the X-ray screening subsystem results in improved sensitivity and specificity.

A further embodiment of the present invention adds a hydrophone for acoustic augmentation of the microwave diagnosis and imaging subsystem.

The present invention reduces the time and equipment costs attendant to existing screening/detection, imaging, and diagnosis/characterization systems and methodologies. In current practice, on average, every 134 X-ray mammogram screenings yield 20 positive detections. Those 20 patients subsequently undergo MRI examinations which indicate, on average, 4 malignant tumors and 16 benign tumors. The four patients with malignant indications undergo invasive biopsies which establish, on average, a single malignancy. In the present invention, ultrasound and microwave modalities replace the role of the MRI examination in the existing art. The ultrasound and microwave methods yield equivalent diagnosis results to MRI at a significantly reduced equipment cost. J. E. Joy, E. E. Penhoet and D. B. Petitti, “Saving Women's Lives: Strategies for Improving Breast Cancer Detection and Diagnosis,” Institute of Medicine and National Research Council, ISBN: 0-309-53209-4, 2005. Further, the ultrasound and microwave methods may be performed in the same examination as the screening X-ray mammogram. This substantially reduces the time and cost associated with multiple separate examinations, and eliminates stress during the time between examinations upon the patient who receives an initial positive indication in the X-ray screening.

Similarly, the present invention provides real-time identification of false positive indications attendant to X-ray mammography. By enabling ultrasound and microwave subsystem diagnosis concurrent with the X-ray mammography screening examination, false positives are quickly identified and dismissed before incurring the stress and costs of further separate examinations.

The present invention enhances screening and diagnosis capability and flexibility by enabling the use of the X-ray, ultrasound or microwave subsystems individually or in combination.

Low cost is achieved by enabling application of low-cost components, such as compact radio frequency components developed for the wireless communications industry and existing ultrasound application components.

In yet another embodiment of the present invention, the complexity, cost and time associated with mechanical scanning is avoided by employing an ultrasound transducer array in place of scanning ultrasound transducers.

The present invention enables three-dimensional screening, imaging and diagnosis. Phased array operation of the ultrasound subsystem allows mapping of two-dimensional planes of varying depths within the target breast. These two-dimensional maps may be integrated to create three-dimensional images.

The present invention eliminates health risks associated with exposure to powerful magnetic fields in MRI, and minimizes the discomfort associated with the long MRI examination times while the patient must hold motionless in the loud and confined MRI apparatus.

Other advantages of the present invention will become readily apparent to those with skill in the art from the following figures, descriptions and claims. As will be appreciated by those of skill in the art, the present invention may be embodied as apparatus, systems or methods. It is intended that such other advantages embodied as other apparatus, systems and methods be included within the scope of this invention, and the examples set forth herein shall not be limiting.

### BRIEF DESCRIPTION OF THE DRAWINGS

The nature of this invention, as well as all its objects and advantages, will become readily apparent and understood upon reference to the following detailed description when considered in conjunction with the accompanying drawings, in which like reference numerals designate like parts throughout the figures thereof, and wherein:

FIG. 1 shows the orientation of the multi-modality system of the present invention with respect to the patient and the imaging target breast in one preferred embodiment of the present invention.

FIG. 2 presents a diagram of the ultrasound subsystem.

FIG. 3 presents a diagram of the microwave imaging subsystem.

FIG. 4 shows the multi-modality system of FIG. 1. FIG. 4a depicts the configuration and operation of the X-ray screening subsystem, the ultrasound subsystem, and the microwave imaging subsystem with respect to the target breast in a preferred embodiment of the present invention. FIG. 4b presents a typical spectral and pictorial display of the output from the combined operation of the ultrasound subsystem and the microwave imaging subsystem.

FIG. 5 shows an alternative embodiment of the present invention wherein the microwave antenna and the ultrasound transducers are oriented on the same side of the breast.

FIG. 6 presents an alternative embodiment of the present invention wherein an acoustic hydrophone is employed in combination with the microwave imaging modality to perform the detection and imaging functions.

FIG. 7 shows an alternative embodiment of the ultrasound subsystem featuring the use of ultrasound arrays. FIG. 7a presents a plan view of a 6×6 ultrasound array. FIG. 7b presents a side view of two 6×6 ultrasound arrays.

FIG. 8 presents an alternative embodiment of the ultrasound subsystem in which a single ultrasound transducer is employed to input integrated ultrasound waves into the target breast. FIG. 8a shows one preferred orientation of the single ultrasound transducer in relation to the target tumor and the microwave antenna. FIG. 8b provides a diagram of the ultrasound subsystem with a single ultrasound transducer.

FIG. 9 presents a diagram of an enhancement to the present invention wherein a closed-loop feedback control system utilizes information from the microwave imaging subsystem to provide input instructions to the ultrasound subsystem.

### DETAILED DESCRIPTION OF THE PREFERRED EMBODIMENTS

The following description is provided to enable any person skilled in the art to make and use the invention and sets forth the best modes contemplated by the inventor of carrying out his invention. Further, while a breast is used in the description of these embodiments, it is to be noted that any turbid medium may be processed with this invention. Thus the present invention shall not be limited to the examples disclosed. The scope of the invention shall be as broad as the claims will allow.

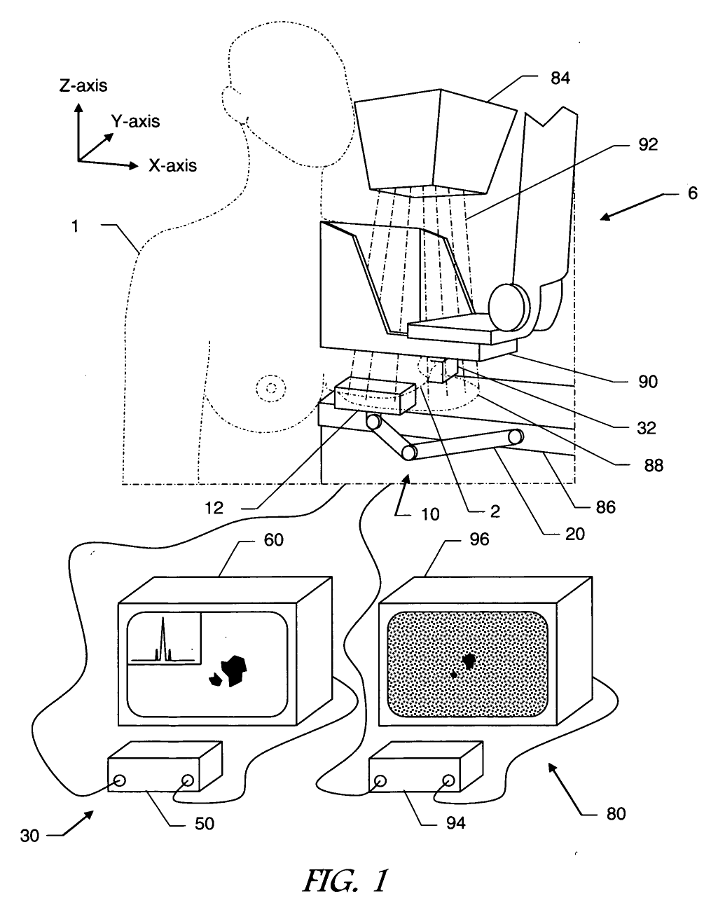

Referring now to the drawings, FIG. 1 shows the orientation of the multi-modality system 6 of the present invention with respect to the patient 1 and the imaging target breast 2 in one preferred embodiment of the present invention. An X-ray screening subsystem 80, an ultrasound subsystem 10 and a microwave imaging subsystem 30 are employed in combination to screen/detect, image and diagnose/characterize tumors in the breast 2. An X-ray detection medium 88 is disposed on the surface of a platform 86. The X-ray detection medium 88 may be any useful medium, typically a film or semiconductor detector array. The target breast 2 is rested on the platform 86 such that the X-ray detection medium 88 is between the platform 86 and the target breast 2. A compression paddle 90 is positioned above the target breast 2 with a downward force sufficient to hold the target breast 2 still during the imaging process and to compress the target breast 2 to enable a consistent image across the target breast 2. An ultrasound electronics assembly 12 and a radiofrequency subsystem 32 are oriented as shown in physical communication with the target breast 2. An X-ray camera 84 produces an X-ray beam 92 which travels through the target breast 2 to the X-ray detection medium 88. Tissues within the target breast 2 having different X-ray absorbing characteristics cause the incident X-ray intensity reaching the X-ray detection medium 88 to vary. The differential exposure of the X-ray detection medium 88 results in the capture of a two dimensional image of the target breast 2, either in the form of exposed film or data from the exposed semiconductor detector array. In the preferred embodiment of the present invention, the X-ray detection medium 88 is a semiconductor detection array which then communicates the sensed X-ray intensity data to a computer/signal and data processor 94. The computer/signal and data processor 94 processes that input data for communication to the technician via a display 96. The display may be a video screen, a printing device, a photographic device, or any useful medium for communicating system output to the technician. The X-ray screening subsystem 80 outputs information regarding the presence and X-Y planar location of suspicious tissues. The technician uses that information to conduct imaging and diagnosis of the target breast in the suspicious areas using the ultrasound subsystem 10 in combination with the microwave imaging subsystem 30.

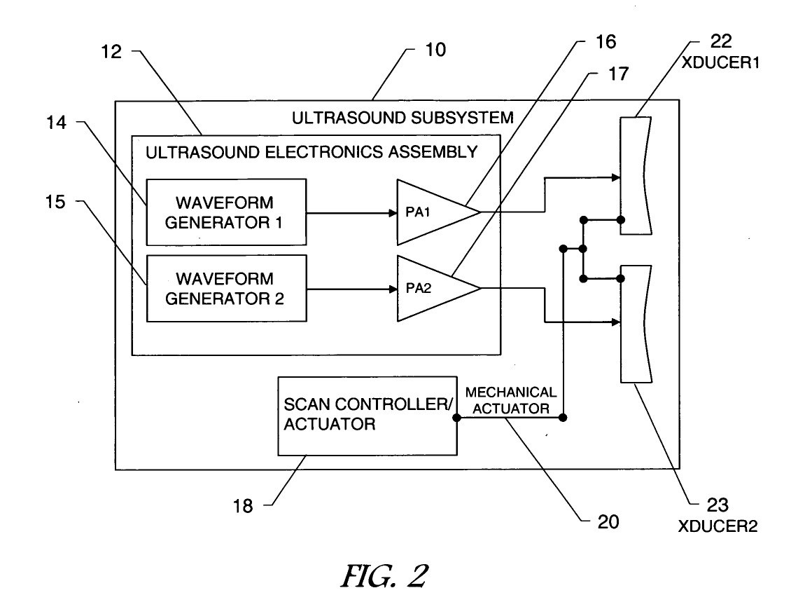

FIG. 2 presents a diagram of the ultrasound subsystem 10. An ultrasound electronics assembly 12 is shown housing a first waveform generator 14 and a second waveform generator 15, and a first power amplifier 16 and a second power amplifier 17. Waveform generator 14 produces a first input ultrasound waveform having frequency f₁. Waveform generator 15 produces a second input ultrasound waveform having frequency f₂. Power amplifier 16 conditions said first input ultrasound waveform and transmits the amplified ultrasound waveform to ultrasound transducer 22. Power amplifier 17 conditions said second input ultrasound waveform and transmits the amplified ultrasound waveform to ultrasound transducer 23. Ultrasound transducer 22 transmits the first amplified input ultrasound wave into the target breast 2. Ultrasound transducer 23 transmits the second amplified input ultrasound wave into the target breast 2. To maximize transmission of said first and second ultrasound waves into the target breast 2, an ultrasound conductive gel may be used at the interface of the ultrasound transducers 22/23 and the target breast 2. In the present embodiment of the invention, the ultrasound transducers 22/23 must be physically relocated to perform a scan of the entire breast 2. This scanning function is performed by a scan controller/actuator 18 working in combination with a mechanical actuator 20. Receiving capability may be added to the ultrasound subsystem 10 to enable use of the ultrasound subsystem 10 as an imaging and diagnosis tool without operation of the microwave imaging subsystem 30.

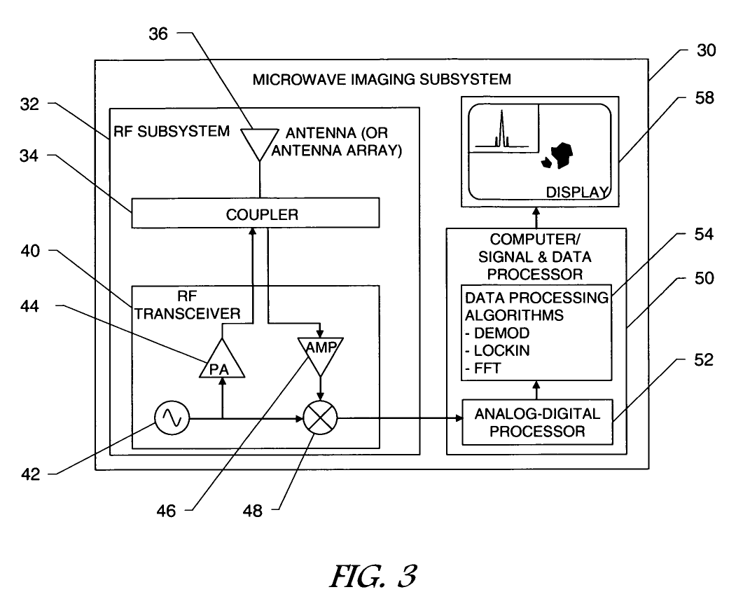

FIG. 3 presents a diagram of the microwave imaging subsystem 30 comprising a radiofrequency (RF) subsystem 32, a computer/signal & data processor 50 and a display 60. The RF subsystem 32 comprises a microwave antenna 36, a coupler 34, and a radiofrequency (RF) transceiver 40. The RF transceiver 40 comprises a waveform generator 42, a power amplifier 44, a linear noise amplifier 46 and a mixer 48. The waveform generator 42 produces an input microwave. The power amplifier 44 conditions the input microwave and transmits said input microwave through the RF coupler 34 to the RF antenna 36. The RF antenna 36 transmits said input microwave into the target breast 2. To efficiently transmit the input microwave into the target breast 2, the RF antenna 36 is in physical communication with the target breast 2. In a preferred embodiment of the present invention, the RF antenna 36 is made from a material that closely matches the dielectric constant of the target breast 2 to enhance transmission into the target breast 2. In an alternative embodiment, a dielectrically loaded antenna, in which the RF antenna 36 is embedded in a material that matches the dielectric constant of the target breast 2, may be employed to reduce reflections. Due to the wide propagation angle of the microwave in the target breast 2, it is not necessary to move the RF antenna to scan the target breast 2. However, an alternative embodiment of the present invention may employ an RF antenna 36 scanning means, if desired. Microwaves reflected by normal and cancerous tissue boundaries and/or inclusions are collected by the RF antenna 36 and transmitted through a coupler 34 to a linear noise amplifier 46. Input microwaves from the waveform generator 42 and reflected microwaves from the linear noise amplifier 46 are passed through a mixer 48 and conveyed to an analog-digital processor 52. Data processing algorithms 54 such as demodulation, and lockin detection or fast Fourier transform algorithms operate on the digital data from the analog-digital processor 52. The resultant frequency and power data is transmitted to a display 60 for viewing by the technician. The display 60 may be a video screen, a printing device, a photographic device, an oscilloscope, a spectrum analyzer, or any useful medium for communicating system output to the technician. The data may be usefully represented as individual spectra, one-dimensional line scans, two-dimensional cross-sectional constructions, or volume images. In the preferred embodiment, the microwave imaging subsystem 30 utilizes continuous wave transmission for the purpose of performing imaging based upon the Doppler effects of the displaced materials. The microwave imaging subsystem 30 may also utilize pulse-delay methodology to enable use of the microwave imaging subsystem 30 as an imaging and diagnosis tool without operation of the ultrasound subsystem 10.

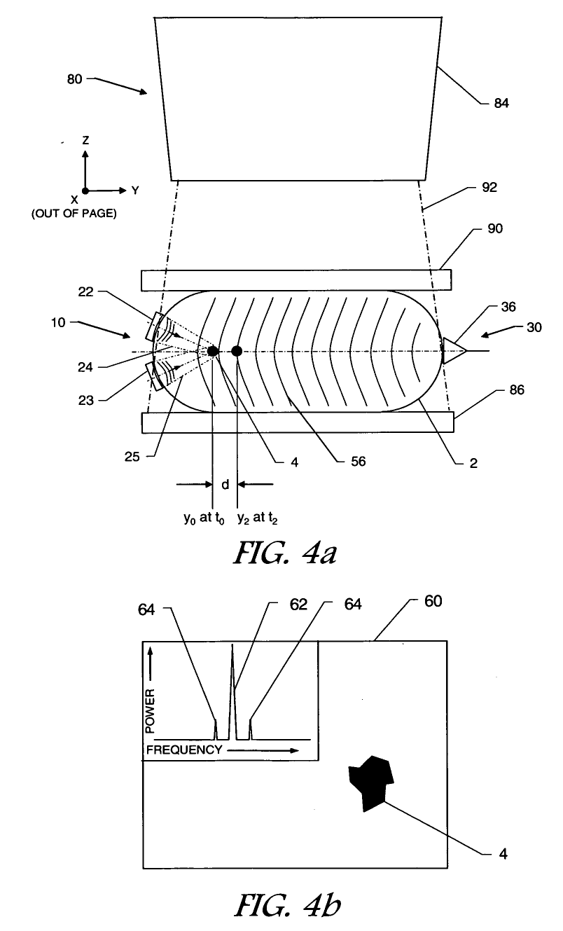

FIG. 4 shows the multi-modality system 6 of FIG. 1. FIG. 4a depicts the configuration and operation of the X-ray screening subsystem 80, the ultrasound subsystem 10, and the microwave imaging subsystem 30 with respect to the target breast 2 in a preferred embodiment of the present invention. FIG. 4b presents a typical spectral and pictorial display of the output from the combined operation of the ultrasound subsystem 10 and the microwave imaging subsystem 30. As discussed in connection with FIG. 1, the information from the X-ray screening subsystem 80 is used to identify areas of interest for further imaging and diagnosis of the target breast 2. The combined operation of the ultrasound subsystem 10 and the microwave imaging subsystem 30 constructs two-dimensional images of planes within the target breast 2. By phased array control of the ultrasound subsystem 10, data is collected for multiple planes at different depths within the target breast 2. These multiple planes are combined to produce three-dimensional images.

According to FIG. 4a, at time t₀, the unexcited tumor 4 is at rest in location y₀ and the microwave antenna 36 is transmitting microwaves 56 into the target breast 2. Prior to activation of ultrasound transducers 22/23, microwaves are reflected back to the microwave antenna 36 from the internal boundaries of the breast 2 and from inclusions in the breast 2 such as a tumor 4. The reflected microwaves are of the same frequency as the transmitted input microwaves 56. As depicted in FIG. 4b, the reflected microwave appears on the display 60 as a power spike 62 at the frequency of the transmitted wave 56. No position or shape information of the tumor 4 is detectable prior to activation of the ultrasound transducers 22/23.

At time t₁, a first ultrasound transducer 22 transmits a first ultrasound beam 24 having a frequency f₁ into the target breast 2, and a second ultrasound transducer 23 transmits a second ultrasound beam 25 having a frequency f₂ into the breast 2. The lenses of the ultrasound transducers 22/23 are designed to create focused ultrasound beams 24/25 which intersect at the target tumor 4. In the preferred embodiment, ultrasound frequencies f₁ and f₂ are high frequencies with a small differential, or beat frequency (f₁-f₂). The high frequencies of the input ultrasound waves 24/25 provide superior resolution and focus capability, but poor tissue displacement force. But as the first and second high-frequency ultrasound waves 24/25 propagate and interact, they produce a series of harmonic waves. One resultant harmonic is a low-frequency wave at the beat frequency (f₁-f₂) resulting from the cancellation of the high-frequency components of the input waves. This low-frequency harmonic component produces a force that excites and displaces the target tissue and tumor 4. Due to the non-linear density and elastic properties of tissues and tumors in the breast 2, the displacement of target tumor 4 can be detected by the microwave imaging subsystem 30. Expressed mathematically:

Source₁=cos(2πf₁t)=cos(ω₁t)

Source₂=cos(2πf₂t)=cos(ω₂t)
Where ω=2πf=angular frequency, and t=time
   Due to high power at the intersection point of the ultrasound beams, non-linearity effects of the tissue become pronounced and the mixing of the two ultrasound signals becomes:

$$\begin{matrix}Resultant = {a}_{1} [ cos ( {ω}_{1} t ) + cos ( {ω}_{2} t ) ] + {{a}_{2} [ cos ( {ω}_{1} t ) + cos ( {ω}_{2} t ) ]}^{2} + … \\ = {a}_{1} cos ( {ω}_{1} t ) + {a}_{1} cos ( {ω}_{2} t ) + {a}_{2} {cos}^{2} ( {ω}_{1} t ) + \\ {a}_{2} {cos}^{2} ( {ω}_{2} t ) + 2 {a}_{2} cos ( {ω}_{1} t )\end{matrix} \\ cos ( {ω}_{2} t ) + … = {a}_{1} cos ( {ω}_{1} t ) + {a}_{1} cos ( {ω}_{2} t ) + {a}_{2} [ 0.5 cos ( 2 {ω}_{1} t ) + 0.5 ] + {a}_{2} [ 0.5 cos ( 2 {ω}_{2} t ) + 0.5 ] + {a}_{2} [ cos ( ( {ω}_{1} + {ω}_{2} ) t ) ) + cos ( ( {ω}_{1} - {ω}_{2} ) t ) ] + …$$

Where a=a constant coefficient dependent upon the non-linearity of the tissue
   The resultant displacement d of the tissue is given by the equation:

d=1(2πf)*sqrt(2FZ)
Where F=energy flux (i.e., power per area),
   Z=tissue acoustic impedance, typically ˜1.5e⁶ kg/m²/s, and
   f=acoustic frequency (in this case f₁-f₂).

Since ω₁ and ω₂ are high frequency to achieve good resolution, then terms with twice the frequency (cos(2ω₁), cos(2ω₁) and cos(ω₁+ω₂)) will be of high frequency and their effect on the motion will be limited. On the other hand, if ω₁ and ω₂ are selected to be close to each other such that (ω₁−ω₂) would be very small (i.e., in order of 100s-1000s Hz), then the term cos((ω₁−ω₂)t) will lead to a large displacement.

At time t₂, the low-frequency ultrasound component impacts the tumor 4 and displaces the tumor 4 to location y₂. The tumor 4 oscillates between location y₂ and y₀ before coming to rest again at essentially the initial location y₀. The ultrasound waves 24/25 travel at a significantly lower rate of speed than the microwave 56. As the tumor 4 oscillates between position y₀ and position y₂, the Doppler effect results in a shift in the frequency of the reflected microwaves. These frequency shifts appear on the display 60 as frequency sidebands 64. Presence of these sidebands indicates the presence of a tumor 4. The sidebands 64 are short lived, essentially lasting for the duration of the ultrasound pulse passing through the tumor 4.

The power of the sidebands 64 is determined through displacement analysis. If a signal is reflected off of a target whose range is changing with time according to r(t)=r₀+Δr(t), the received signal can be written as:

s(t)=cos[ω_{c}t+2π−Δr(t)/λ+φ₀]

Where ω_{c} is the carrier frequency and ω₀ is the phase

For a small-amplitude oscillation of a target with a displacement d and a modulation frequency fm, the range is given by:

Δr(t)=d sin(ωₘt)

And thus the signal becomes

s(t)=cos[ω_{c}t+2π−(d/λ)sin(ωₘt)+φ₀]

For d<<λ, this expression is simply the narrowband FM situation:

$$\begin{matrix}f ( t ) = cos [ {ω}_{c} t + ( d / λ ) sin ( {ω}_{m} t ) ] \\ = cos ( {ω}_{c} t ) cos ( ( d / λ ) sin ( {ω}_{m} t ) ) - sin ( {ω}_{c} t ) sin ( ( d / λ ) sin ( {ω}_{m} t ) ) \\ = cos ( {ω}_{c} t ) - ( d / 2 λ ) [ cos ( {ω}_{c} t - {ω}_{m} t ) - cos ( {ω}_{c} t + {ω}_{m} t ) ]\end{matrix}$$

Each sideband is smaller than the carrier by:

P_{sideband}=10 log(d²/4λ²)=20 log(πf_{c}d/c) dBc.

Radio frequency sensitivity is determined by the equation:

Sensitivity=NF+KT+10 log(BW)+SNR−10 log(Average)

### Where

NF: The receiver input referred noise figure (Typically 3-5 dB)

KT: Thermal noise power density (−174 dBm/Hz)

BW: Receiver noise bandwidth in Hz (typically 1-2 MHz)

SNR: Required detector SNR in dB (20 dB)

Average: Coherently collected samples over sample time

If sensitivity is not sufficient, and to give system sensitivity a boost, a continuous wave may be employed such that:

Sensitivity=NF+KT+10 log(BW)+SNR−10 log(Average)−10 log(gain)

### Where

gain: gain achieved due to applying continuous wave

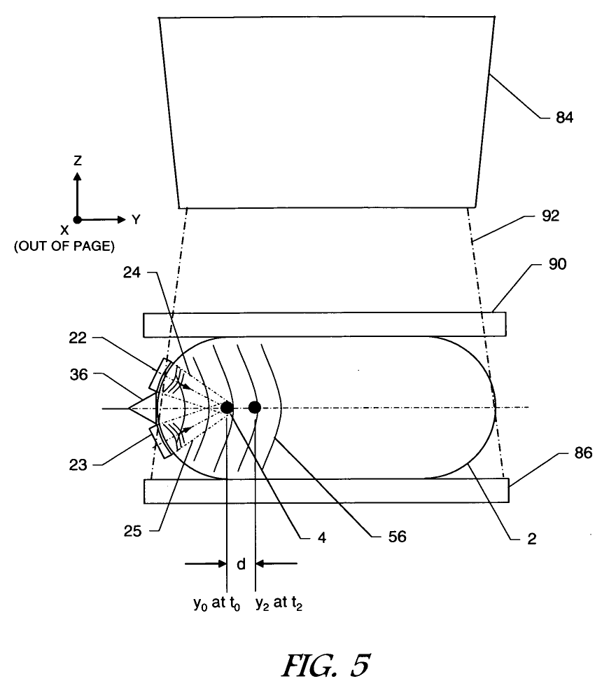

FIG. 5 shows an alternative embodiment of the present invention wherein the microwave antenna 36 is oriented on the same side of the breast as the ultrasound transducers 22/23. The concept of operation and the method of use are identical to that of the embodiment of FIG. 4, but this alternative may provide packaging advantages over that embodiment.

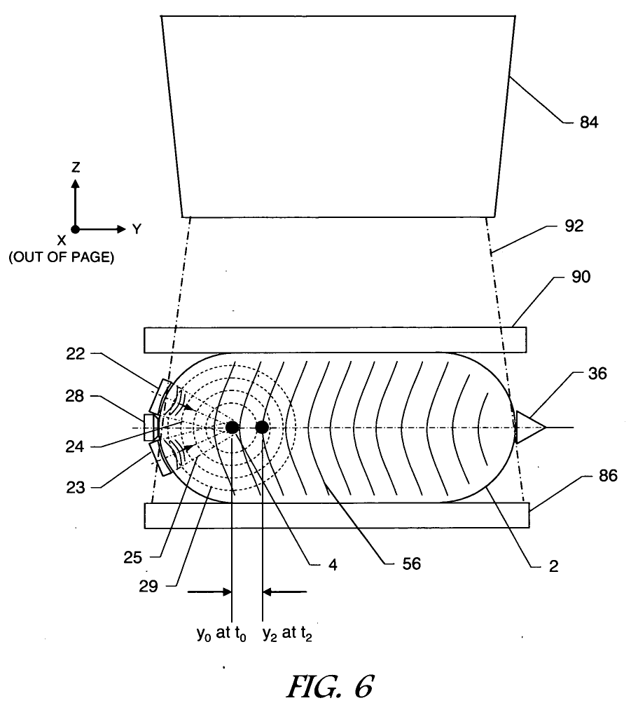

FIG. 6 presents an alternative embodiment of the present invention wherein an acoustic hydrophone 28 is employed in combination with the microwave imaging modality to perform diagnosis and imaging functions. The acoustic hydrophone 28 receives sonic waves 29 generated by the motion induced in the tissue by the ultrasound beams 24/25. The displacement data collected by the acoustic hydrophone 28 may be combined with the high-contrast, high-specificity data collected by the microwave modality to augment the diagnosis function.

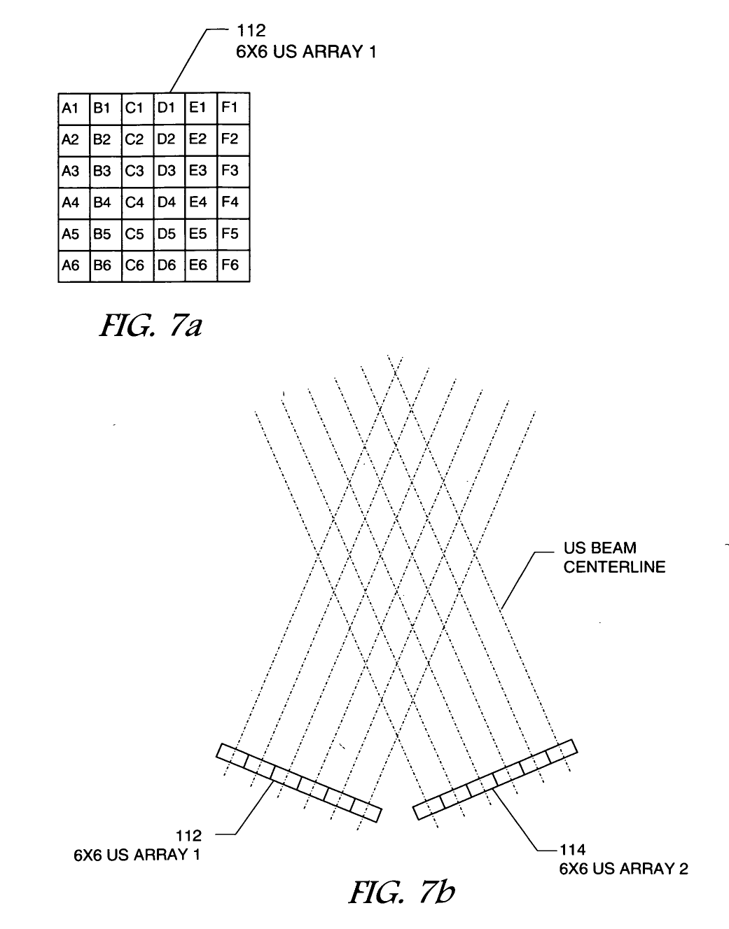

An alternative embodiment of the present invention employs ultrasound arrays 112/114 in place of the scanning ultrasound transducers 22/24. FIG. 7 illustrates an exemplary ultrasound array implementation. FIG. 7a presents a plan view of a 6×6 ultrasound array 112 with ultrasound transducer elements arranged in a matrix of rows 1 through 6 and columns A through F. FIG. 7b presents a side view of two 6×6 ultrasound arrays 112/114. The two ultrasound arrays operate in cooperation to transmit ultrasound waves into the breast. By selectively activating ultrasound elements of each of the arrays, the paired arrays 112/114 can focus input ultrasound energy waves at the intersection of the ultrasound beam centerlines of the activated ultrasound elements. Further, electronic tuning of the dual-array system permits focus between the centerline intersection points. This embodiment permits detection to be performed throughout a large volume of the breast without the need for scanning. This embodiment trades the physical complexity and longer examination time associated with scanning implementations for the greater electronic implementation complexity of the ultrasound array implementation. While a symmetrical 6×6 array is shown to illustrate the concept, many array configurations may be usefully employed. 3×120 and 5×120 non-symmetrical arrays and 1×120 linear arrays are found in literature.

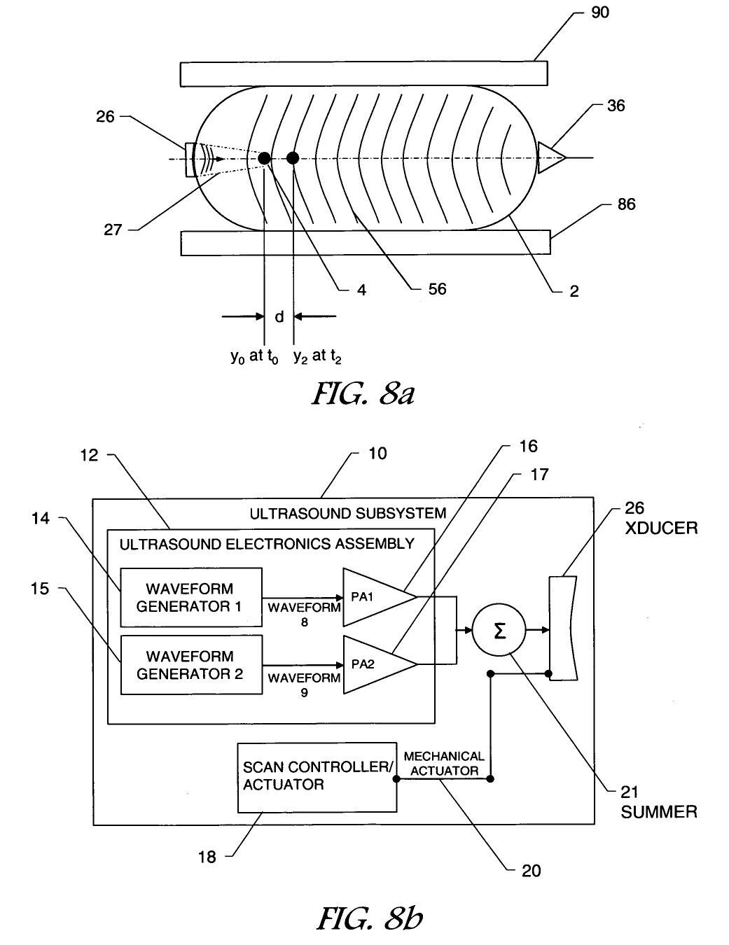

FIG. 8 presents an alternative embodiment of the ultrasound subsystem 10 in which a single ultrasound transducer 26 is employed to input multiple ultrasound waves integrated into a single ultrasound beam 27. FIG. 8a presents one preferred embodiment of this alternative wherein the microwave antenna 36 and the ultrasound transducer 26 are positioned on opposite sides of the breast 2. An alternative embodiment positions a single annular ultrasound transducer around the microwave antenna 36 such that the transducer and antenna are on the same side of the breast 2. FIG. 8b provides a diagram of the ultrasound system 10 with a single ultrasound transducer 27. In this embodiment, waveform generator 14 produces a first input ultrasound waveform 8 having frequency f₁. Waveform generator 15 produces a second input ultrasound waveform 9 having frequency f₂. A first power amplifier 16 conditions the first input ultrasound waveform 8 and transmits said first ultrasound waveform 8 to a summer 21. A second power amplifier 17 conditions the second input ultrasound waveform 9 and transmits said second ultrasound waveform 9 to the summer 21. The summer 21 combines said first and second input ultrasound waveforms 8/9 and transmits the combined waveforms 8/9 to a single ultrasound transducer 26. The ultrasound transducer 26 transmits the combined input ultrasound waves 8/9, in a single, integrated focused ultrasound beam 27 comprising both f₁ and f₂ components, into the breast 2. As discussed relative to the embodiment of FIG. 4, the combined high-frequency ultrasound waves 8/9 propagate and interact to produce a series of harmonic waves. One resultant harmonic is a low-frequency wave at the beat frequency (f₁-f₂). This low-frequency harmonic component produces a force that excites and displaces the target tumor 4.

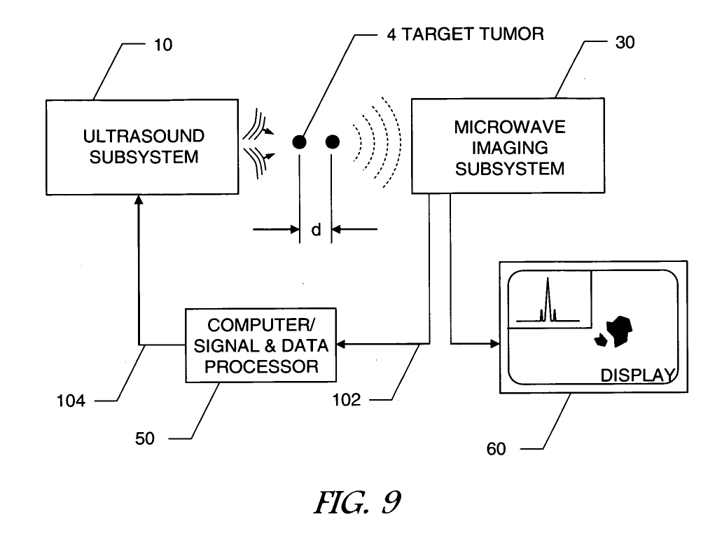

FIG. 9 presents a diagram of an enhancement to the present invention wherein a closed-loop feedback control system utilizes information from the microwave imaging subsystem 30 to provide input instructions to the ultrasound subsystem 10 thereby enhancing the efficiency, accuracy and quality of the screening and diagnosis process. The ultrasound subsystem 10 excites the region of interest resulting in displacement d of the target tumor 4. The microwave imaging subsystem 30 detects microwaves reflected by the excited target tumor 4. The microwave imaging subsystem 30 processes the detected microwaves into detection, diagnosis and imaging information which is made available to the technician by means of the display 60. Microwave imaging subsystem information 102 is further processed by means of a computer/signal and data processor 50 into ultrasound subsystem input instructions 104 which are communicated to the ultrasound subsystem 10. Scanning process efficiency may be enhanced by providing real time instructions such as desired scanning regions, patterns and depths. Image quality may be controlled by providing real time instructions relative to resolution, contrast, power levels, continuous waveform versus pulse, pulse rate, drift compensation/stability, accuracy, noise and scan velocity.

Various embodiments of the present invention may be exercised in ways other than illustrated in the examples shown in the Figures. Such alternative embodiments are within the contemplation of the present invention. The examples are not intended to limit the scope of this invention, which shall be as broad as the claims will allow.

In addition, the present invention may be adapted to a variety of applications in both medical and non-medical fields. The field of medical soft tissue imaging includes orthopedics, dermatology, breast tumor screening/detection, imaging and diagnosis/characterization, and other medical applications. Such alternative applications are within the contemplation of the present invention and the scope of the invention shall be as broad as the claims will allow.

The physical implementation of the present invention may be varied without departing from the spirit of the invention. Elements and components may be implemented, added, interchanged, combined and/or packaged in a variety of embodiments. Various changes may be effected in structure, design, choice of components and materials, etcetera without departing from the spirit of the present invention. Such alternative embodiments, elements and implementations are within the contemplation of the present invention.

Accordingly, the scope of the invention should be determined not by the embodiments illustrated, but by their legal equivalents, and shall be as broad as the claims will allow.

The following references are helpful in understanding the foregoing specification and are incorporated herein by reference:

Li, Xu, et al. (2004): Microwave Imaging via Space-Time Beamforming: Experimental Investigation of Tumor Detection in Multilayer Breast Phantoms, IEEE Transactions on Microwave Theory and Techniques, Vol. 52, No. 8, pp 1856-1865.

Nanda, R. (2007): “Breast Cancer,” Medline Plus Medical Encyclopedia, the U.S. National Library of Medicine and the National Institute of Health, <http://www.nlm.nih.gov/medlineplus/ency/article/000913.htm>.

A. Alizad, M. Fatemi, L. E. Wold and J. F. Greenleaf, “Performance of Vibro-Acoustography in Detecting Microcalcifications in Excised Human Breast Tissue: A Study of 74 Tissue Samples,” IEEE Trans. Med. Imaging., vol. 23, pp. 307-312, March 2004.

C. Maleke, J. Luo and E. E. Konofagou, “2D Simulation of the Harmonic Motion Imaging (HMI) With Experimental Validation,” IEEE Ultrasonics Symposium, pp. 797-800, 2007.

E. E. Konofagou, M. Ottensmeyer, S. L. Dawson and K. Hynynen, “Harmonic Motion Imaging—Applications in the Detection of Stiffer Masses,” IEEE Ultrasonics Symposium, pp. 558-561, 2003.

“Mammography,” Radiology Info, Radiological Society of North America (RSNA), pp. 6-7, 2006, <http://www.radiologyinfo.org/en/info.cfm?pg=mammo&bhcp=1>.

J. E. Joy, E. E. Penhoet and D. B. Petitti, “Saving Women's Lives: Strategies for Improving Breast Cancer Detection and Diagnosis,” Institute of Medicine and National Research Council, ISBN: 0-309-53209-4, 2005.

W. A. Berg, et al., “Combined Screening With Ultrasound and Mammography vs Mammography Alone in Women at Elevated Risk of Breast Cancer,” Journal of the American Medical Association (JAMA), vol. 299 No.18, pp. 2151-2163, May 14, 2008.

What is claimed is:

1. A system for screening, diagnosing and imaging of materials and objects in a dense compressive media comprising:
(a) a first means for screening and imaging materials and objects in the media employing an X-ray screening means in combination with;
(b) a second means for screening, diagnosing and imaging of objects in the dense compressive media further comprising:
(i) an ultrasound means for exciting regions, materials and objects in the dense compressive media employing a plurality of ultrasound wave beams in combination with
(ii) a microwave means for detecting, characterizing and imaging the excited materials and objects.

2. The system according to claim 1, wherein
(a) said X-ray screening means comprises:
(i) a means for generating input X-ray radiation;
(ii) a means for exposing the dense compressive media to the input X-ray radiation;
(iii) a means for detecting the X-ray radiation that passes through the dense compressive media; and
(iv) a means for communicating and displaying the detected pass-through radiation;
(b) said ultrasound means comprises:
(i) a means for generating a plurality of input ultrasound waves having small differential frequencies; and
(ii) a means for transmitting said plurality of ultrasound waves into the dense compressive media; and
(c) said microwave means comprises:
(i) a means for generating input microwaves;
(ii) a means for transmitting said input microwaves into the dense compressive media;
(iii) a means for detecting microwaves reflected by boundaries, materials and objects in the dense compressive media;
(iv) a means for processing detected microwaves into information describing the presence, location and characteristics of the materials and objects; and
(v) a means for communicating and displaying said information.

3. A method for screening, diagnosis and imaging of materials and objects in a dense compressive media comprising the steps of:
(a) screening and imaging materials and objects in the media utilizing an X-ray screening and means;
(b) utilizing information collected by the X-ray screening means to identify regions for further screening, diagnosis and imaging by ultrasound means in combination with microwave means;
(c) exciting regions, materials and objects in the dense compressive media by transmitting a plurality of ultrasound wave beams into the region; and
(d) screening, diagnosing and imaging the excited materials and objects employing microwave means.

4. A system for screening, diagnosis and imaging of materials and objects in a dense compressive media comprising:
(a) an X-ray screening subsystem for screening and imaging materials and objects in the media in combination with;
(b) an ultrasound subsystem for generating a plurality of ultrasound waves for exciting materials and objects in the dense compressive media; and
(c) a microwave imaging subsystem for screening, diagnosis and imaging said excited materials and tissues.

5. A method for screening, diagnosis and imaging of materials and objects in a dense compressive media comprising the steps of:
(a) performing X-ray screening and imaging of the media;
(b) utilizing information collected by the X-ray screening and imaging to identify regions for further screening, diagnosis and imaging by ultrasound means in combination with microwave means;
(c) generating input microwaves;
(d) transmitting said microwaves into the dense compressive media;
(e) generating a plurality of input ultrasound waves;
(f) transmitting said input ultrasound waves, and the resultant low-frequency, high displacement force beat frequency ultrasound waves, into the dense compressive media to excite materials and objects in the media;
(g) detecting microwaves reflected by the excited materials and objects;
(h) converting said detected microwaves into information describing the presence, location and characteristics of the excited materials and objects; and
(i) displaying said information.

6. A system for screening, diagnosis and imaging of materials and objects in a dense compressive media comprising:
(a) an X-ray screening subsystem further comprising:
(i) an X-ray camera for exposing the media to an X-ray beam,
(ii) an X-ray detection medium such as a semiconductor detection array to detect X-ray radiation passing through the media,
(iii) a computer/signal and data processor for processing detected X-ray radiation into information describing the presence, location and characteristics of materials and objects in the media, and
(iv) a display for communicating the information.
(b) an ultrasound subsystem further comprising:
(i) a plurality of waveform generators to produce ultrasound waveforms of differential frequency,
(ii) a plurality of power amplifiers to condition the generated ultrasound waveforms,
(iii) a plurality of ultrasound transducers to transmit the conditioned ultrasound wave into the target media and excite materials and objects within the media, and
(iv) a scan controller/actuator to enable scanning of the media; and
(c) a microwave imaging subsystem further comprising:
(i) a microwave generator for producing microwaves,
(ii) a power amplifier to condition the generated microwaves,
(iii) a microwave antenna or antenna array to transmit the conditioned microwave into the target media and to detect microwaves reflected by media boundaries and materials within the media,
(iii) a computer/signal and data processor for processing detected analog microwave signals into information describing the presence, location and characteristics of the excited materials and objects, and
(iv) a display for communicating the information.

7. The system according to claim 6, wherein the plurality of ultrasound transducers is embodied in an ultrasound transducer array.

8. The system according to claim 6, wherein a single ultrasound transducer transmits the plurality of ultrasound waves into the media.

9. The system according to claims 1, 2, 4 or 6, further comprising an acoustic means of detecting sonic waves generated by said excited materials and objects in the dense compressive media.

10. The system of claim 9 wherein said acoustic means is a hydrophone.

11. The system according to claims 1, 2, 4 or 6, further comprising a feedback control system for utilizing output information from the microwave means to generate and communicate instructions to the ultrasound means.

12. A method of using the system according to claims 1, 2, 4 or 6, in which the X-ray screening subsystem, the ultrasound subsystem and the microwave imaging subsystem may be used either singly or in combination.
